(19) United States
(12) Patent Application Publication
(10) Pub. No.: US 2006/0190021 A1
(43) Pub. Date: Aug. 24, 2006

(54) Cutting device for subcutaneous incisions

(76) Inventors: Michael Hausman, New York, NY (US); Michael Gregory Thomas, Van Alstyne, TX (US); Brian David Adams, Lowa City, IA (US); John R. Pepper, Cheshire, CT (US)

Correspondence Address: SOFER & HAROUN, LLP, Suite 910, 317 Madison Avenue, New York, NY, 10017, US

(21) Appl. No.: 11/061,769

(22) Filed: Feb. 18, 2005

(51) Int. Cl. A61B 17/32

(52) U.S. Cl. 606/167

(57) ABSTRACT

A cutting device is provided for making subcutaneous incisions. The cutting device has a housing forming a handle. A tubular blade sub-assembly extends from the housing. A trocar passes through the tubular blade sub-assembly and is configured to be positioned in a first active position, with a tip of the trocar extending beyond the end of the tubular blade sub-assembly and a second retracted position where the tip of the trocar is held within the end of tubular blade sub-assembly.

### FIELD OF THE INVENTION

The present invention relates to a cutting device for safely dividing tissues using a minimally invasive surgical approach. More particularly the present invention relates to a cutting device for use in surgically treating certain conditions requiring a subcutaneous incision, such as trigger finger.

### BACKGROUND OF THE INVENTION

The medical condition, commonly referred to as trigger finger, is a common cause of hand pain and hand disability. The condition usually manifests as pain when moving the digits or may manifest acutely as a painful popping or snapping as the patient moves the affected digit. In more severe cases, the condition may result in partial or complete locking of the digit in the flexed position, requiring physical manipulation to return the digit to full extension.

In the hand, a number of flexor tendons run across the palm, and then up along each of the digits. These flexor tendons are responsible for aiding in the flexing of the digits. Each of the flexor tendons is contained within a sheath which runs from the tips of digits down to the metacarpal heads at the palm of the hand. A series of pulleys, both annular and cruciate, are formed in the sheath to assist in the flexing of the digits. As the digit is flexed, the flexor tendon slides within the sheath, through the pulleys. When a particular pulley is inflamed for any reason, such as volar flexor tenosynovitis, the increased thickness of the pulley hinders the movement of the underlying flexor tendon, causing the above described condition. In fact, in more serious conditions, the pulley inflammation can be to such an extent that it partially deforms the underlying flexor tendon, further exacerbating the condition.

There are a number of manners to treat this condition currently in use. The first and most common treatment is direct injection of steroids in the area of the affected pulley. The steroid injection is used to lessen the inflammation of the pulley so as to allow the flexor tendon to move more freely, alleviating the triggering condition. Although this treatment is effective in many cases, sometimes the steroids fail to reduce the inflammation sufficiently to alleviate the condition.

When the steroid treatment fails, the current method to treat the condition is to surgically divide the affected pulley. However, there are many drawbacks to this treatment. The first and most obvious is that there is significant scaring and healing times, as is the case in most surgeries. In order to reach and cut the affected pulley, the skin over the affected area needs to be opened sufficiently and the subcutaneous tissue over the sheath needs to be separated or cleared. Because the pulley itself may be up to a 1 cm in width (perpendicular to the axis of flexion), the area that needs to be accessed is significant enough to generate scaring which can greatly affect healing time. This is particularly true, given that the affected area is located in a region that has difficulty healing, given its proximity to flexing joint. Another drawback to this surgical method is that it is time consuming, requiring wound and suture care, bandage, etc.

A second surgical method is to use endoscopic surgery in order to minimize the invasiveness of the surgery. Using this method, the incision in the hand near the pulley is minimal, reducing the amount of scar tissue. However, when performing any endoscopic surgery, there is a need to use the related equipment. The equipment needed to perform an endoscopic surgery is expensive and may not be available for a surgery as simple as cutting a pulley to relieve trigger finger.

### OBJECT AND SUMMARY OF THE INVENTION

The present invention looks to overcome the drawbacks associated with the prior art methods of treating trigger finger by providing a cutting device for making a subcutaneous incision for treating the condition of trigger finger that significantly reduces the amount of scar tissue by reducing the size the of necessary incision, while simultaneously providing a simple, rapid and cost effective method for performing the surgery, possibly in a private office, without the need for using expensive and difficult to obtain endoscopic equipment.

To this end the present invention provides for a cutting device for safely dividing tissues beneath the skin without requiring direct exposure and visualization of the structure to be cut. The cutting device includes a housing forming a handle. A tubular blade sub-assembly extends from the housing. A trocar passes through the tubular blade sub-assembly, and is configured to be positioned in a first active position, with a tip of the trocar extending beyond the end of the tubular blade sub-assembly and a second retracted position where the tip of the trocar is held within the end of tubular blade sub-assembly.

### BRIEF DESCRIPTION OF THE DRAWINGS

The subject matter regarded as the invention is particularly pointed out and distinctly claimed in the concluding portion of the specification. The invention, however, both as to organization and method of operation, together with features, objects, and advantages thereof may best be understood by reference to the following detailed description when read with the accompanying drawings in which:

FIG. 1 is a front perspective view of a cutting device, in accordance with one embodiment of the present invention;

FIG. 2A is a top view of the cutting device shown in FIG. 1, in accordance with one embodiment of the present invention;

FIG. 2B is a side view of the cutting device shown in FIG. 1, in accordance with one embodiment of the present invention;

FIG. 3 is an expanded perspective view of the cutting device shown in FIG. 1, in accordance with one embodiment of the present invention;

FIG. 4, is a close up cut away view of a trocar locking mechanism, in accordance with another embodiment of the present invention;

FIG. 5 is a close up cut away view of the trocar locking mechanism from FIG. 4, in accordance with another embodiment of the present invention;

FIG. 6 is a close up cut away view of the trocar locking mechanism from FIG. 4, in accordance with another embodiment of the present invention;

FIG. 7 is a close up cut away view of the trocar locking mechanism from FIG. 4, in accordance with another embodiment of the present invention;

FIG. 8 is a close up view of a cutting head with the trcoar in the retracted position, in accordance with one embodiment of the present invention;

FIG. 9 is a close up view of a cutting head with the trocar for ward in the active position, in accordance with one embodiment of the present invention;

FIG. 10 is a representation of the anatomy of a digit showing the region of operation for the device of FIG. 1 in accordance with one embodiment of the present invention;

FIG. 11 is a flow chart of an exemplary surgical procedure using the device of FIG. 1, in accordance with one embodiment of the present invention;

FIG. 12 is an image of an insertion step in a trigger finger release surgery showing the cutting device from FIG. 1, in accordance with one embodiment of the present invention;

FIG. 13 is a close up view of a cutting head with the trcoar in the retracted position showing the placement of the cutting head relative to the sheath, in accordance with one embodiment of the present invention;

FIG. 14A is a is a side view of the cutting device shown in FIG. 1 with an optional depth stop indicator, in accordance with one embodiment of the present invention; and

FIG. 14B is a close up the depth stop indicator from FIG. 14A, in accordance with one embodiment of the present invention.

### DETAILED DESCRIPTION

In accordance with one embodiment, the present invention is directed to a cutting device 10 for making subcutaneous incisions, used for surgically treating conditions, such as trigger finger. Although the following description of device 10 is discussed in terms of treating the trigger finger condition, this is done by way of example, and is in no way intended to limit the scope of the present invention. For example, device 10 or a variation thereof may be used for a number of surgical treatments that require a subcutenaeous incision. Such other uses may include but are not limited to division of any retinacular structure, including, but not limited to, the transverse carpal ligament, medial or lateral retinaculum of the knee or elbow, release of an extensor or flexor retinaculum of the wrist and plantar fascia.

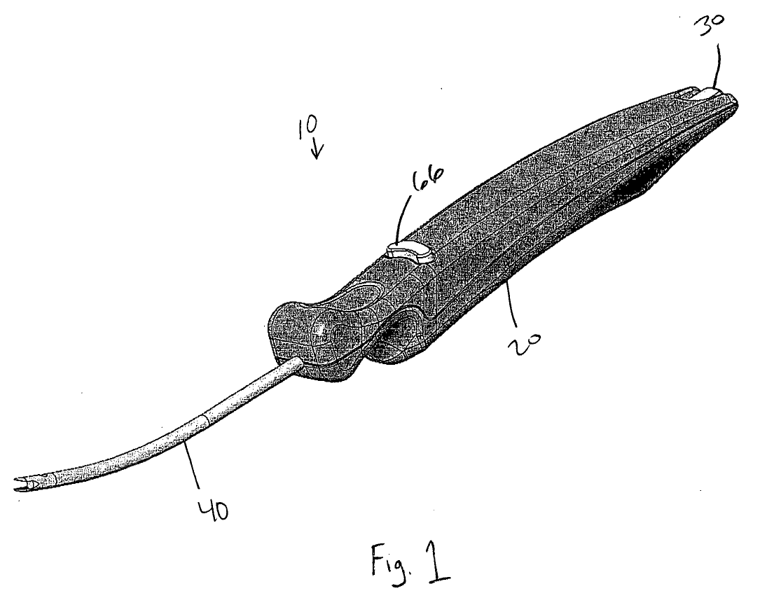

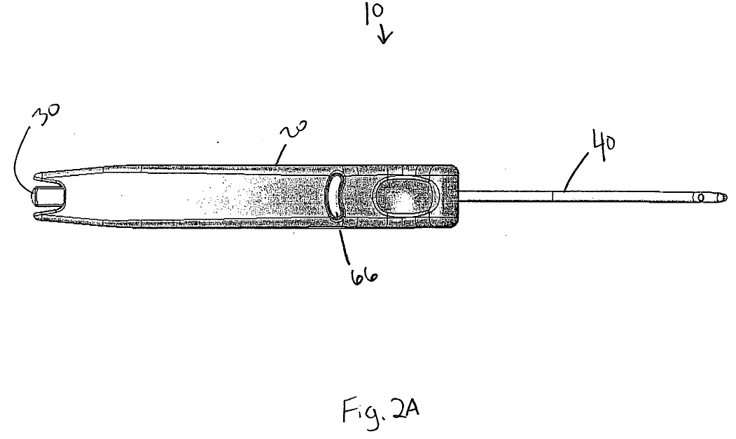

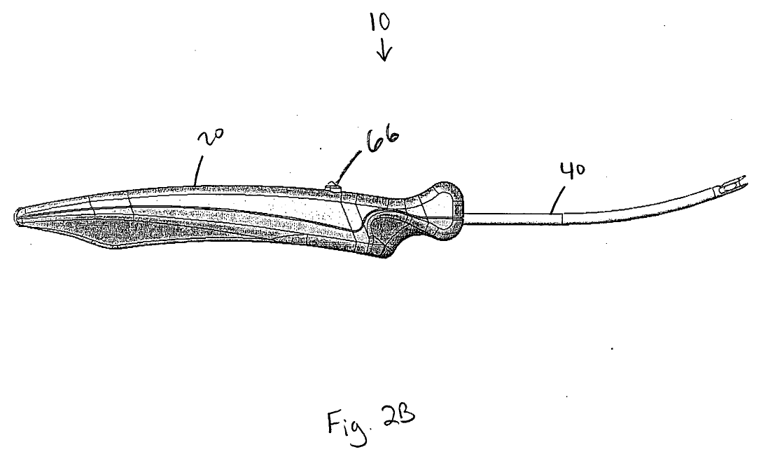

Device 10, as illustrated in FIG. 1, is comprised of a housing sub-assembly 20, a trocar sub-assembly 30 and a tubular blade sub-assembly 40. FIG. 1 also shows release portion 66 of locking button 60, the function of which is described in more detail below. A top view of device 10 is shown in FIG. 2A, and a side view is shown in FIG. 2B, also illustrating the sub-assemblies relationship with one another.

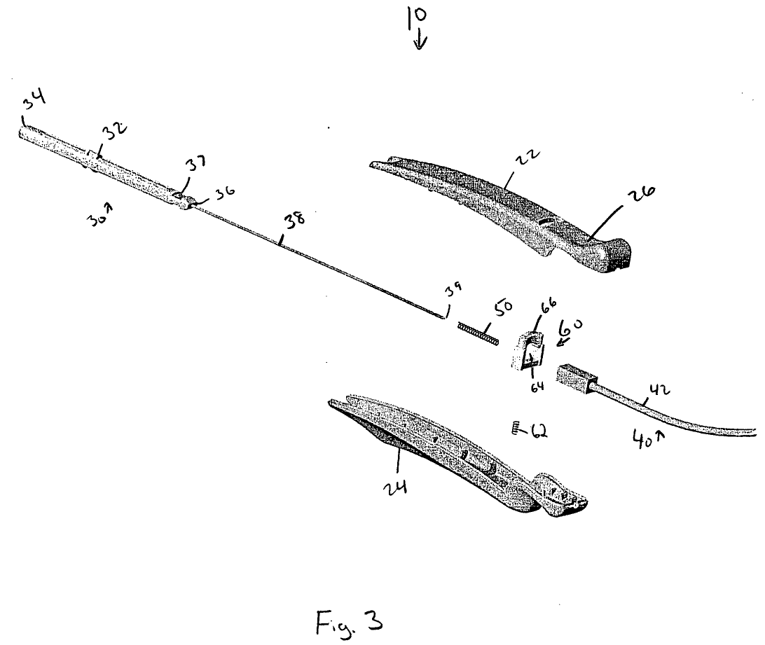

Turing to FIG. 3, an expanded view of device 10 is shown, displaying the full view of each of the sub-assemblies 20, 30 and 40. Beginning with housing sub-assembly 20 (also refereed to as housing 20), there is a top portion 22 and bottom portion 24 which are placed together to form the handle for the device. Top portion 22 and bottom portion 24, may be either removably snapped together or permanently fixed to one another based on the manufacturer's requirements. Both top portion 22 and bottom portion 24 are preferably constructed of ABS plastic (Acrylonitrile-Butadiene-Styrene), however, other materials may also be used such as polymers, plastics, epoxy resins or stainless steel (surgical grade).

Housing 20, when formed together, preferably has a contoured surface, also illustrated in FIGS. 2A and 2B, that comfortably matches the hand of the surgeon. The design shown is for use in either hand, but left and right handed designs as well as larger and smaller handles are also within the contemplation of the present invention. However, for the purposes of illustration, the current design, discussed throughout, is for use in with hand.

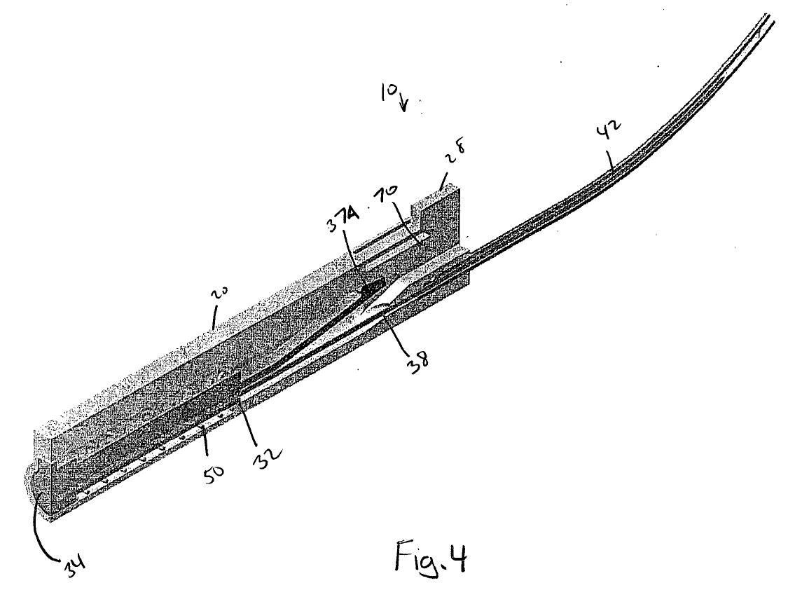

Top portion 22 of housing 20, further maintains a thumb imprint 26, configured to conform to the thumb of the surgeon so as to provide additional grip strength during the surgery and to prevent the hand from slipping off of device 10. Also, as described in more detail below, with respect to trocar sub-assembly 30, housing 20 may also maintain an opening to allow a trocar release button 60 to pass therethrough. Alternatively, a deformable trocar release area 28 as an integrally formed feature of housing 20 as discussed below and shown in FIGS. 4-7.

Trocar sub-assembly 30 (also referred to as trocar assembly 30), as illustrated in FIG. 3, is comprised of trocar base 32 and the trocar 38. Trocar base 32 is preferably constructed of a polypropylene plastic. However, is can be construed of any similar material such as ABS plastics, polymers, plastics, epoxy resins or stainless steel (surgical grade).

Trocar base 32, maintains a trocar activation button 34, a front plate 36 and a trocar lock 37. Trocar activation button 34 is a portion of trocar base 32 that extends out of the back end of housing 20. Trocar activation button 34 allows a user to manipulate the trocar to a forward, active or spring loaded position, as will be discussed in more detail below.

Front plate 36 is a front portion of trocar base 32, out of which trocar 38 exits towards tubular blade sub-assembly 40. Trocar lock 37 is a portion of trocar base 32 that is used to lock trocar base 32 and the attached trocar 38 in a forward active position within housing 20.

Trocar 38 is a flexible metal or fiber wire that extends out of front plate 36 of trocar base 32 and into tubular blade sub-assembly 40. In the medical device industry a trocar generally refers to an extended usually cylindrical implement with a pointed, but blunted tip. In the present invention, trocar 38, is preferably constructed of nitinol (Nickel Titanium Navel Ordinance Laboratory). However, in other preferred embodiments, any similar flexible metal that is similar in strength and flexibility may also be used.

Trocar 38 terminates in a rounded conical or semi/frustoconical trocar tip 39. Alternatively, trocar tip 39 may be a beveled non-conical tip. In either arrangement, trocar tip 39 must be sufficiently smaller than the thickness of trocar 38 so that it is capable of penetrating the flexor tendon sheath, while simultaneously being blunt enough so that it provides a distinctive tactile and/or auditory feedback when the retinacular structure is entered by trocar 38 and such that trocar 38 does not do any extraneous damage below the sheath 304, such as cutting of the underlying flexor tendon 302, as discussed in more detail below. The function of the blunted tip 39 is also more fully described in the surgical process example discussed below.

Trocar 38 is manipulated through housing 20 into a first forward or active position and a second retracted position. The first active position refers to a forward trocar 38 position, whereby trocar tip 39 is extended beyond the end of tubular blade sub-assembly 40 and the second retracted position refers to a retracted trocar 38 position, whereby trocar tip 39 is returned to a stored position entirely within tubular blade sub-assembly 40. A detailed description of these positions is discussed in more detail below.

Trocar lock 37, used to maintain these two forward and retracted positions, is discussed hereafter. As illustrated in FIG. 3 a spring 50 is positioned within housing 20, so as to contact trocar base 32, and is configured to bias trocar base 36 into a retracted rearward position.

In a first embodiment, as illustrated in FIG. 3, trocar lock 37 is configured as a reduced thickness notch in trocar base 32. In the retracted position, trocar base 32 is biased in a rearward direction within housing 20, exposing trocar activation button 34 out of the rear of housing 20.

In this arrangement a locking button 60 is provided which is biased in an upward position by a biasing spring 62. Locking button 60 maintains a central opening 64 which allows the front of trocar base 32 to pass therethrough. Locking button 60 further maintains a release portion 66 that extends out of the top of housing 20 through a hole in upper portion 22.

When the surgeon wishes to move trocar 38 to a forward active position, the trocar activation button 34 is pushed forward from the rear of trocar base 32, forcing trocar 38 forward through tubular blade sub-assembly 40. As trocar base 32 is pushed forward, the front of base 32 passes through central opening 64, depressing locking button 60 momentarily. As trocar base 32 is pushed in far enough forward, biasing spring 64 continues to force button 60 upward until it locks trocar lock 37 in trocar base 32. In this position, trocar 38 is locked in the forward active position.

To release trocar 38, the surgeon can simply press down on release portion 66 of locking button 60, forcing it downward to the point where central opening 64 no longer holds against trocar lock 37. Once removed from locking button 60, spring 50 biases trocar base 32 back to its original retracted position, forcing trocar tip 39 back inside of the end of tubular blade sub-assembly 40.

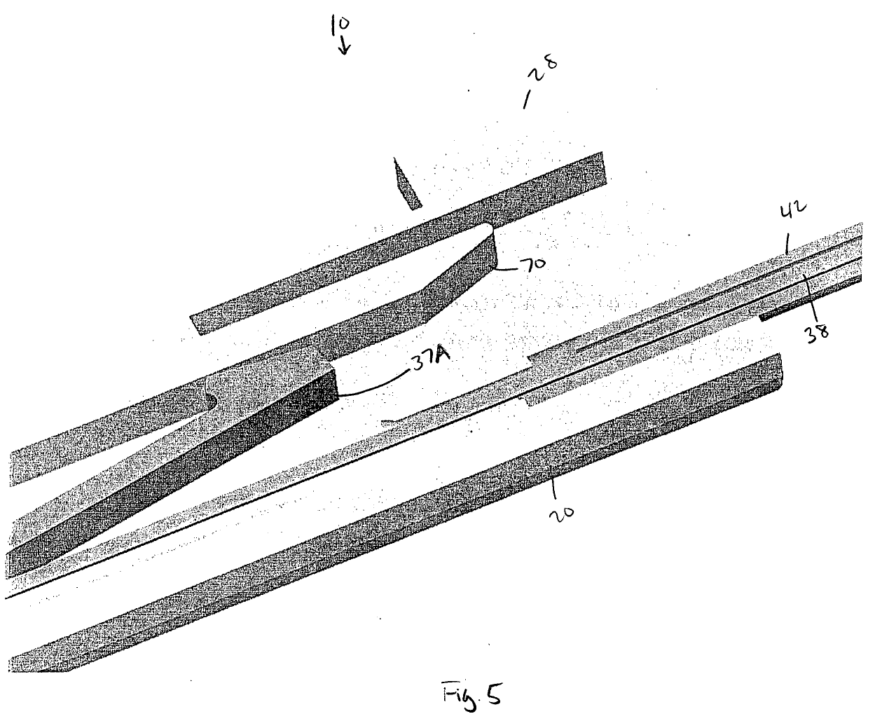

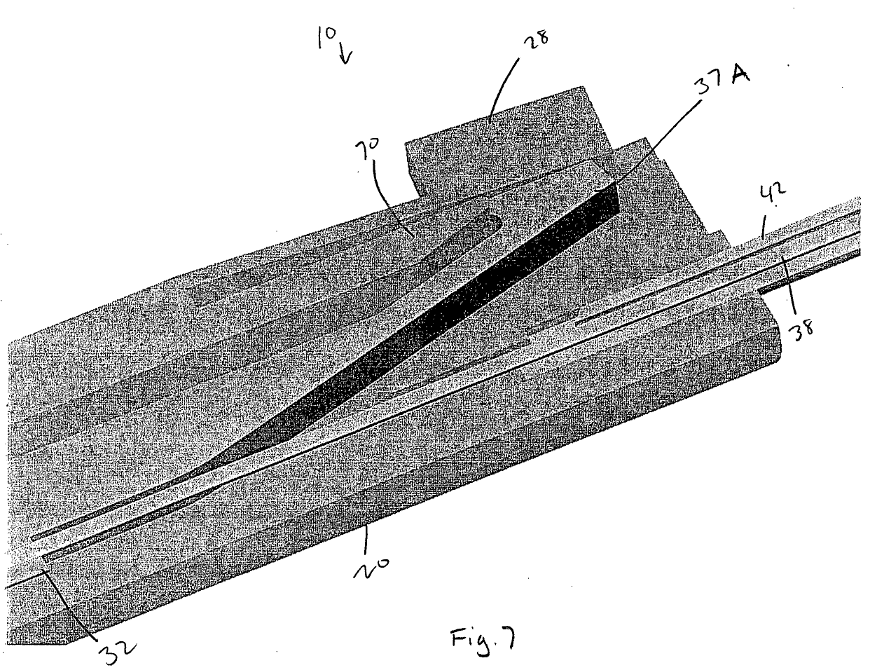

In another embodiment of the present invention, a second arrangement is shown in FIGS. 4, 5, 6 and 7 where trocar lock 37A is in the form of a raised locking hook extending from trocar base 32. As seen in the cutaway view FIG. 7, of housing 20, in this configuration, trocar lock 37A is formed as a raised locking hook extending from trocar base 32 configured to catch a latch 70, formed as part of top portion 22 of housing 20 in order to lock trocar 38 in the forward active position. As with the above described embodiment shown in FIG. 3, spring 50 is normally set to bias trocar base 32 back to its inactive retracted position. FIG. 7 also illustrates a deformable trocar release area 28 as an integrally formed feature of housing 20. FIG. 5 illustrates a close up view of latch 70, trocar lock 37A and trocar release area 28 in the inactive retracted position.

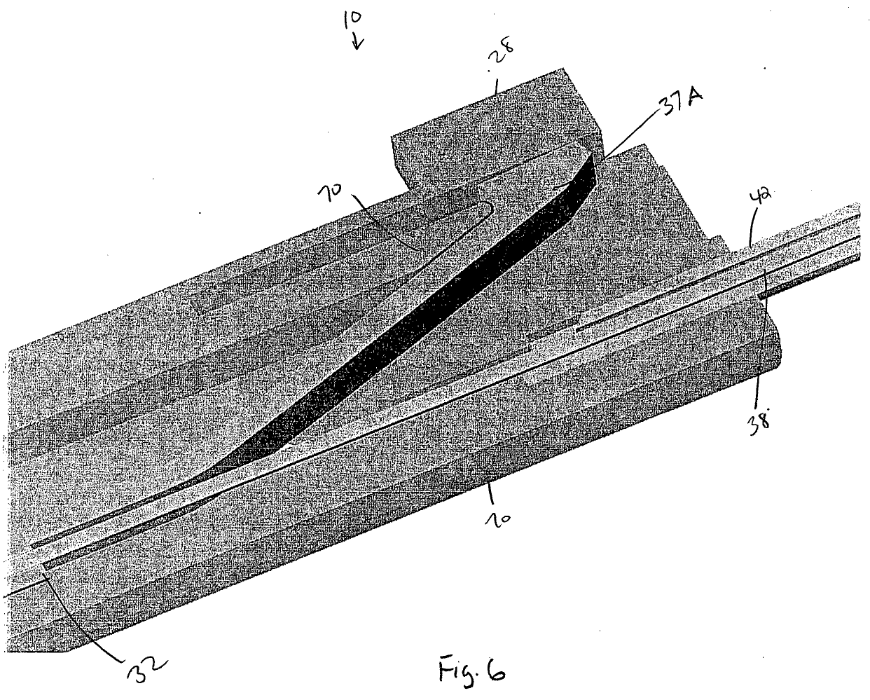

To activate trocar tip 39 beyond the end of tubular blade sub-assembly 40, the surgeon presses trocar activation button 34 forward, forcing trocar base 32 forward. As trocar base 32 moves forward, trocar lock 37A, in the form of the locking hook, catches on latch 70 as illustrated in close up FIG. 6, locking trocar 38 in the forward active position.

To release trocar 38 to the retracted position, with trocar tip 39 contained back within the end of tubular blade sub-assembly 40, the surgeon presses on deformable trocar release area 28. As noted above, trocar release button 28 is formed in housing 20 as a plastically deformable tab, as shown in FIG. 7, that deforms downward pushing trocar lock 37A away from latch 70, allowing spring 50 to bias trocar base 32 back into the inactive retracted position within housing 20.

It is understood that these two arrangements for moving trocar 38 between the active and retracted positions are intended only as examples of possible constructions of device 10, and are in no way intended to limit the scope of the invention. For example, in another embodiment of the invention, a manual lever may be added to trocar assembly 30, that extends out of housing 20, allowing the surgeon to move trocar 38 from a retracted to a forward/active position, rather than using the spring loaded versions described above. Any similar device 10, having a trocar moving between a forward and retracted position is within the contemplation of the present invention.

Returning to the basic construction of device 10, as illustrated in FIG. 3, tubular blade sub-assembly 40 (also referred to as blade assembly 40) is generally constructed as a hollow metal tube 42 that extends out of the front end of housing 20, having a cutting head 80 at the end away from housing 20, and is configured to provide a conduit for trocar 38.

Tubular blade sub-assembly 40 is pictured in FIG. 3 as having an ABS plastic base however, this is in no way intended to limit the scope of the invention. Blade assembly 40 may be attached in any manner to housing 20 that provides the requisite sturdiness for performing the surgery, without blade assembly 40 falling off of or loosening from housing 20.

Tubular blade sub-assembly 40 is preferably constructed of surgical grade stainless steel, but it may also be constructed of any other metal that is capable of holding a blade edge and meets the other necessary criteria for surgical metals or of a plastic or composite in which a suitable cutting edge 88 can be embedded or attached. Blade subassembly 40 may be tubular shaped, but may also be rectangular, triangular or any other polygonal of complex volume form.

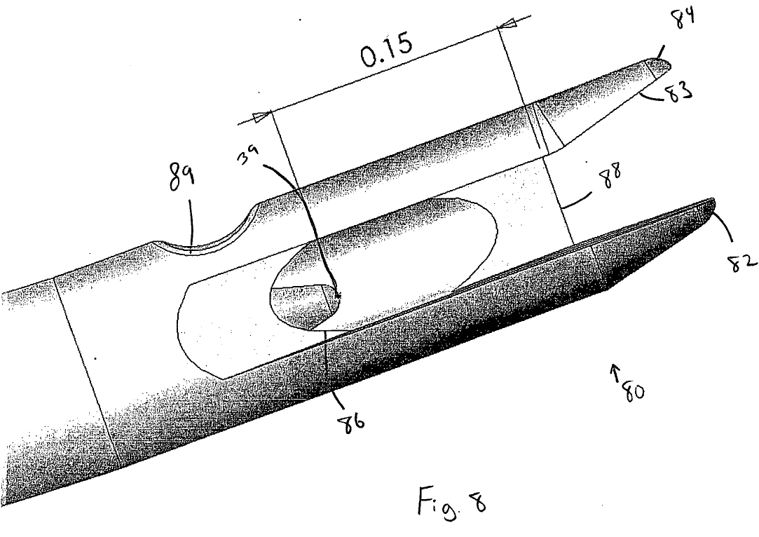

In a first retracted arrangement, as illustrated in FIG. 8, a close up illustration of cutting head 80, located at the end of trocar blade sub-assembly 40, is provided showing trocar tip 39 all the way within tubular assembly 40 in the inactive retracted position. Cutting head 80 includes a first lower guiding edge 82, a second angled upper guiding edge 84, a recess cutaway 86 and a cutting edge 88. As with all of blade assembly 40, the elements of cutting head 80 are all preferably constructed of surgical grade stainless steel.

Lower guiding edge 82 and upper angled guiding edge 84 are both tapered and polished to smooth rounded edges. The under side of upper angled guiding edge 84 has a smooth deflection face 83 on its lower side. Cutting edge 88 is a sharpened blade/edge that is used to perform the necessary subcutaneous cuts described below. Cutting edge 88 is preferably disposed substantially vertically between and substantially perpendicular to first lower guiding edge 82 and second upper guiding edge 84. Cutting edge 88 may be a straight edge perpendicular to the long axis of blade sub-assembly 40 or may be angled or crescent-shaped.

Cutting edge 88 is shown in FIG. 8 on a first left side of the end of tubular assembly 40, however the invention is not limited in this respect. Cutting edge 88 may be on either the left or the right side of tubular assembly 40, between lower guiding edge 82 and upper guiding edge 84.

As illustrated in FIG. 8, recess cutaway 86 is a blunt edge, similar to that found on lower guiding edge 82 and upper guiding edge 84, allowing cutting edge 88 to be the sole forward edge on the lateral sides of cutting head 80. As shown in FIG. 8, recess cutaway 86 is located on the right lateral side of tubular assembly 40, between lower guiding edge 82 and upper guiding edge 84, opposite cutting edge 88. As noted above, cutting edge 88 may be placed on the right lateral side of tubular assembly 40, in which case recess cutaway 86 would likewise be switched to the opposing left lateral side. In either arrangement, cutting edge 88 and recess cutaway 86 are always on opposing lateral sides of tubular assembly 40.

In this configuration, with cutting head 80 having only a single forward cutting edge 88, with the other side of metal tube 42 being a recess cutaway 86, device 10 is able to cut a single incision without the other lateral side of cutting head 80 interfering with the cut, or, from producing a second unwanted cut on the opposing lateral side. It is noted that recess cutaway 86 is preferably cut back along tubular assembly 40 to a distance significant enough so as not to interfere with the cutting operations of cutting edge 88 while simultaneously being cutaway to a small enough extent so as not to prevent cutting end 80 of tubular assembly 40 from properly guiding trocar tip 39 of trocar 38 or to allow undesirable deformation of the upper and lower guides 82 and 84.

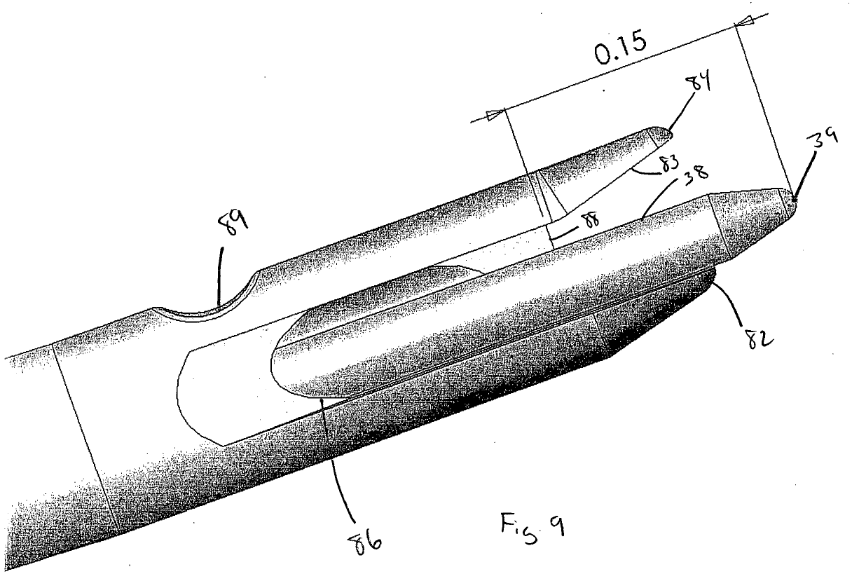

As shown in FIG. 9, cutting head 80 also maintains a dimple 89, configured to press trocar 38 and trocar tip 39 downward so that as it exits cutting head 80 of blade assembly 40 it is pressed flush against lower guiding edge 82, away from upper angled guiding edge 84. In this arrangement, dimple 89 is formed in cutting head 80 of tubular assembly 40 to an extent enough to press against trocar 38, while simultaneously allowing trocar 38 to pass smoothly and unimpeded between the forward active and rearward inactive positions described above.

The junction between trocar 38 and lower guide edge 82 of cutting head 80 is tapered, chamfered and polished to minimize drag on the tissues so as to permit trocar 38 to easily guide lower guide edge 82 of cutting head 80 into sheath 304 or any other retinacular space. This configuration of graduated diameter differentials from trocar tip 39, trocar 38 and lower guide edge 82 of cutting head 80 allows for easy penetration of the retinacular structure by trocar 38 and lower guide egde 82, thus guiding the larger diameter of cutting head 80 of tubular blade sub-assembly 40 into the correct position, while minimizing the risk of “wandering” or sliding of cutting head 80 into an undesirable and incorrect position.

Angled upper guide edge 84 of cutting head 80 is splayed and angled to provide a sudden change in diameter relative to the rear end of cutting head 80 and tubular blade sub-assembly 40 that traps device 10, preventing over-penetration and thus ensures that cutting edge 88 of cutting head 80 is inserted to the appropriate depth. As trocar 38 is retracted, the retinacular structure to be cut is thus trapped and constrained between the splayed angled upper guide edge 84 and lower guide edge 82 of cutting head 80 which can be advanced only until the sharpened cutting edge 88 meets the resistance of the retinacular structure, such as the A1 pulley 300.

As such, a device 10 is provided for use in subcutaneous surgical procedures. The following is an exemplary surgical procedure using device 10 in a trigger finger release surgery, for cutting a pulley in the hand, for example, the A1 pulley.

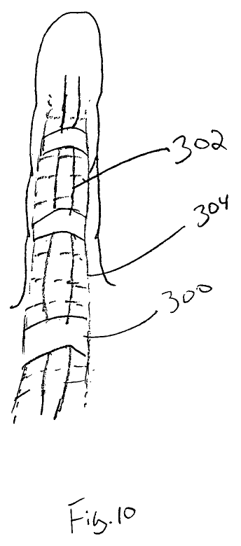

As illustrated in, FIG. 10, the general anatomy of the A1 pulley area of the patient's hand includes A1 pulley 300, flexor tendon 302 and sheath 304. Flexor tendon 302 runs through sheath 304 along the length of the finger. Pulleys, including A1 pulley 300 are bands of thickened sheath that assist in keeping the tendon in place during finger flexion. As the finger bends, flexor tendon 302 slides through sheath 304, under the pulleys including A1 pulley 300.

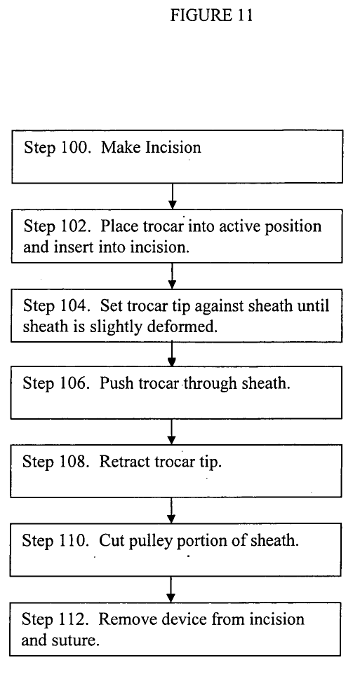

In one embodiment of the present invention as shown in flow chart FIG. 11, a procedure is described for performing a subcutaneous surgical procedure whereby A1 pulley 300 is cut to release the underlying flexor tendon 302. As noted above, this surgery is intended only as example of one use for device 10 and is in no way intended to limit the scope of the invention. Any similar subcutaneous surgical procedure using a similar device 10 is also within the contemplation of the present invention.

In a first step 100, the surgeon locates a point just above or just below (along the axis of the digit) the desired pulley to be cut, such as A1 pulley 300, and an incision is made under local anesthetic. This incision may be made vertically along the flexion axis of flexor tendon 302, or horizontally, perpendicular to the axis of flexor tendon 302. The incision may be made above the affected pulley, such as A1 pulley 300, directly on the affected digit, or it may even be made below the pulley on the affected digit or in the palm, near the metacarpal base of the digit. It is noted that typically such surgeries are performed in a proximal to distal direction. Device 10 of the present invention may be used in this direction, however because of its unique design it is able to work in the distal to proximal direction as well, from above the affected pulley.

In the present example, set forth in flow chart 11, the incision for cutting A1 pulley 300 is being made from above pulley 300, along the affected digit. Because cutting head 80 and trocar tip 39 are small, the necessary incision need only be big enough to allow cutting head 80 to enter below the skin. This cut may be as small as 2-4 mm, significantly less than is need to perform the surgery manually, without endoscopic equipment, such open procedure normally requiring 1-2 cm or more for the incision.

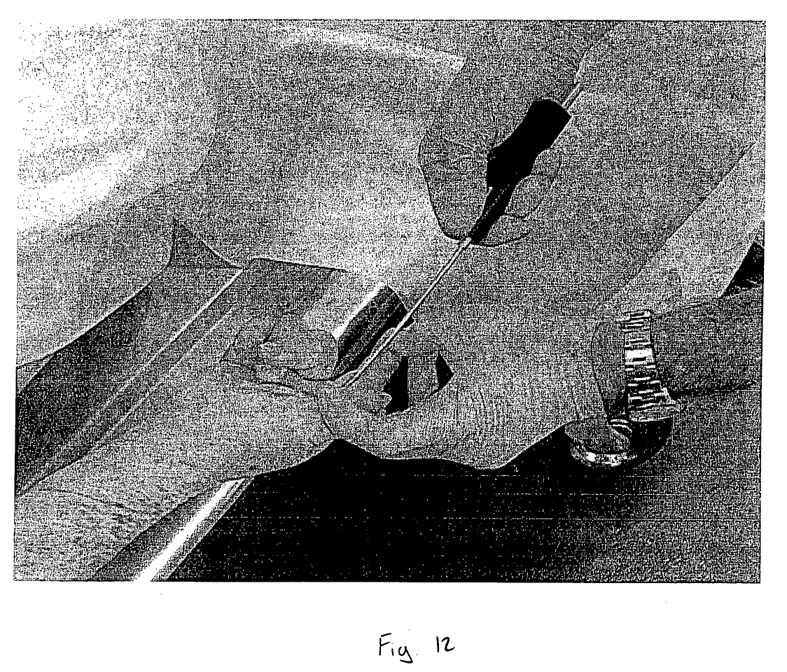

Next, at step 102, the surgeon places trocar 38 in the forward or active position as shown above in FIG. 8. Once in the active position, cutting head 80 of device 10, with trocar tip 39 exposed, is placed into the incision. A picture of cutting head 80 and trocar tip 39 entering the incision is shown in FIG. 12.

At step 104, once inside the incision, the surgeon locates the point at which they desire to enter sheath 304. By using trocar tip 39, the surgeon, deforms a portion of sheath 304 to hold device 10 in the proper position. The surgeon then advances trocar 38 into sheath 304 with an audible and tactile popping sensation due to the semi-blunt bevel of trocar tip 39. Trocar tip 39 is pointed enough to not slip off the convex pulley, such as A1 pulley 300, but is not so sharp as to easily cut into the pulley without additional effort and without a definite sense of tactile feedback confirming that trocar 38 has penetrated sheath 304. As described above, trocar tip 39 is sufficiently dull not to simply cut through sheath 304 and underlying flexor tendon 302, but is simultaneously pointed enough to slightly deform the outside of sheath 304. If tip 39 were insufficiently pointed, it would simply slip off of sheath 304.

Next, at step 106, once trocar tip 39 is set into the proper location, the surgeon presses down on device 10, pushing trocar tip 39 into sheath 304. This procedure is typically accompanied by a popping sound or sensation, signifying that trocar tip 39 has penetrated sheath 304.

As noted above trocar 38 and trocar tip 39 are flush against lower guiding edge 82 of cutting head 80. As such, as trocar tip 39 penetrates sheath 304, lower guiding edge 82 also enters sheath 304. However, as cutting head 80 is advanced further into sheath 304, deflection face 83 of upper angled guide edge 84 contacts the outside/top surface of sheath 304, resulting in upper and lower guide edges 84 and 82 straddling the sheath.

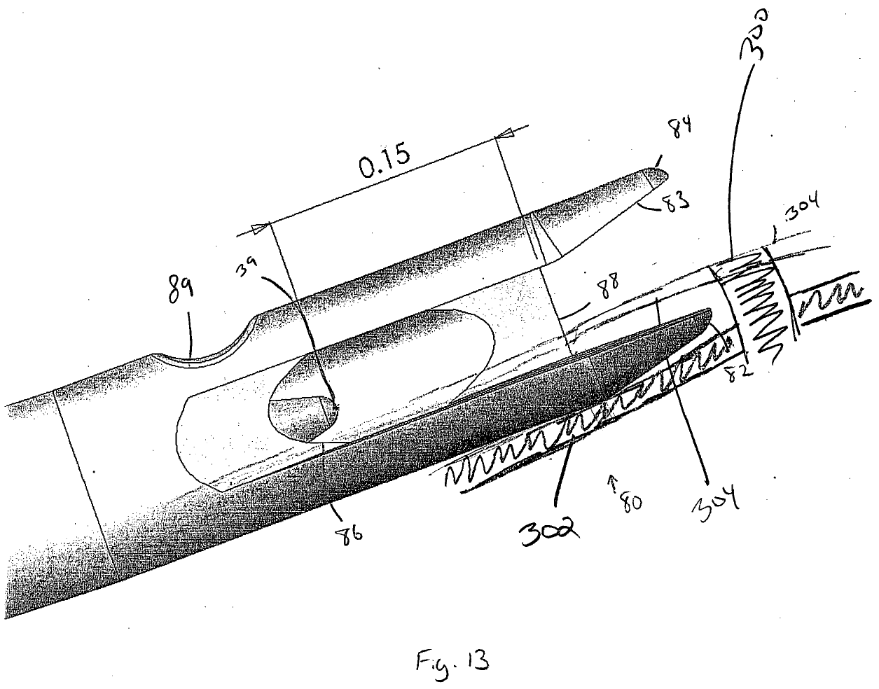

Once inside sheath 304, at step 108, trocar tip 39 is retracted according to the above described process, leaving lower guiding edge 82 inside sheath 304 and upper angled guiding edge 84 on the outside (above the top surface) of sheath 304. In this arrangement, cutting edge 88 is now aligned to make the incision downward into the affected pulley, such as A1 pulley 300, portion of the sheath 304, as illustrated in FIG. 13.

Next, at step 110, the surgeon pushes device 10 towards A1 pulley 300, forcing cutting edge 88 of cutting head 80 to cut through therethrough. The surgeon cuts until the affected pulley is entirely opened. Advancement of device 10 with gentle pressure allows sharpened cutting edge 88 of cutting head 80 to divide the tissue constrained within upper and lower guide edges 84 and 82. Upper and lower guide edges 84 and 82 are blunt and polished and thus designed to follow a path of least resistance, thus correctly guiding the cutting edge 88 of cutting head 80 along the retinaculum and resisting deviation into adjacent structures. The curved, blunt, edge of upper angled guide edge 84 dissects adjacent tissue off the retinaculum, protecting these structures. The biconvexity, or other complex shape, of upper guide edge 84 also maximizes its “presentation surface” or area for the given volume of material, thus optimizing the “plowing” and retracting effect for the given size of the structure.

The blunt slot or recessed cutaway 86 of cutting head 80 opposite cutting edge 88 allows the cut edge of the retinacular tissue to deflect off and past cutting head 80 without resistance, so as not to impair the tactile feedback from cutting edge 88 to the operator's hand.

This distance for this cut may be confirmed in two ways. First, there is a definite change in the tactile feel of the instrument as the resistance to forward motion lessens once the stiff tissue of the pulley is cut and the cutting assembly advances into different, more compliant tissue. This process of finishing the cut is usually determined once the resistance to the cut from sheath 304 reduces significantly, indicating that cutting edge 88 is now all the way past the region of A1 pulley 300 in sheath 304.

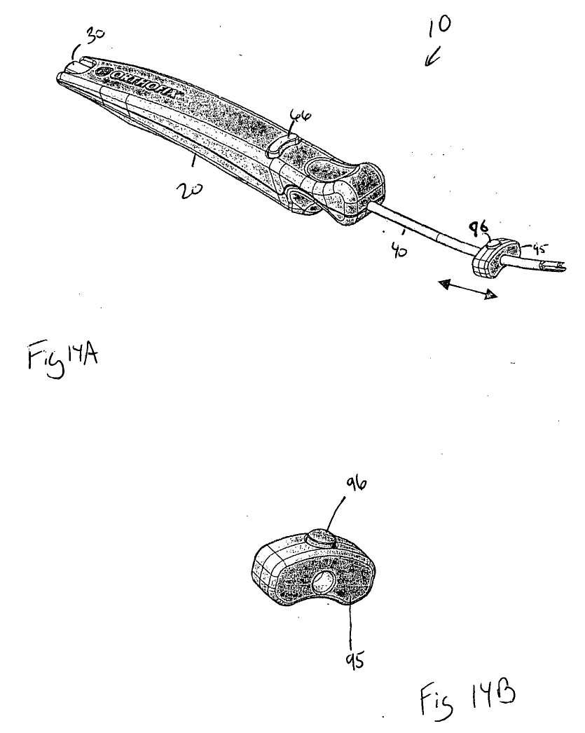

Secondly, as the approximate dimensions of the pulley are known from anatomical studies, a depth gauge (or depth markings) on the cutting assembly confirm the depth of the cut. In a first arrangement, (not shown), the depth gauge may be simply and etched line along cutting head 80 or tubular blade sub-assembly 40. In an alternative arrangement, illustrated in FIGS. 14A and 14B, a moveable depth gauge 95 may be used. Moveable depth gauge 95 is preferably kidney bean shaped, having the same contour as the patient's finger, but may take any other form of similar size. A release/lock gauge button 96 is disposed on top, allowing the surgeon to set the distance manually prior to surgery. This is particularly advantageous when using the same device 10 for different types of operations, other than A1 pulley release.

Finally, at step 112, device 10 is removed from the digit and the incision is sutured.

Again, as noted above, the description of this operation is intended as one example, of a possible surgery conducted with device 10. However, any similar subcutaneous surgical procedure, using a similar device 10, is within the contemplation of the present invention.

While only certain features of the invention have been illustrated and described herein, many modifications, substitutions, changes or equivalents will now occur to those skilled in the art. It is therefore, to be understood that this application is intended to cover all such modifications and changes that fall within the true spirit of the invention.

What is claimed is:

1. A cutting device for making subcutaneous incisions, said cutting device comprising:
a housing, said housing forming a handle;
a tubular blade sub-assembly extending from said housing; and
a trocar, said trocar passing through said tubular blade sub-assembly, configured to be positioned in a first active position, with a tip of said trocar extending beyond the end of said tubular blade sub-assembly and a second retracted position wherein said tip of said trocar is held within the end of tubular blade sub-assembly.

2. The cutting device as claimed in claim 1, wherein said housing is constructed of plastic and is contoured to a user's hand.

3. The cutting device as claimed in claim 1, wherein said tubular blade sub-assembly is constructed of surgical stainless steel.

4. The cutting device as claimed in claim 1, wherein said tubular blade sub-assembly maintains a cutting head, said cutting head further comprising a first lower guide edge and a second upper angled guide edge and a cutting edge on a first lateral side.

5. The device as claimed in claim 1, wherein said cutting edge is disposed between said first lower guide edge and said second upper angled guide edge in any one of straight perpendicularly between said first and second guide edges, angled between said first and second guide edges and crescent shaped between said first and second guide edges.

6. The cutting device as claimed in claim 4, wherein said cutting head further comprises a blunt cutaway on a second lateral side opposite said cutting edge, wherein said cutaway is configured to allow cut tissues to glide with minimal resistance along said device.

7. The cutting device as claimed in claim 4, wherein said cutting head further comprises a dimple on an upper side, configured to depress said trocar downward, so that when said tip of said trocar exits said cutting head, it is pressed downward, flush against said first lower guide edge.

8. The cutting device as claimed in claim 4, wherein said first lower guide edge is tapered, chamfered and polished to a rounded end.

9. The cutting device as claimed in claim 8, wherein said tip of said trocar is a rounded to either one of a non-sharpened or semi-sharpened tip.

10. The cutting device as claimed in claim 9, wherein said tip of said trocar is conical or frustoconical in shape.

11. The cutting device as claimed in claim 9, wherein said tip of said trocar is a beveled non-conical shape.

12. The cutting device as claimed in claim 9, wherein the combined shape of first lower guide edge and said tip of said trocar as said tip of said trocar exits said cutting head pressed downward flush against said first lower guide edge, is such that said combined shape of first lower guide edge and said tip of said trocar form a substantially single continuous implement of graduated diameters

13. The device as claimed in claim 12, wherein said second upper angled guide edge is splayed upward on said cutting head away from said first lower guide edge and is tapered, chamfered and polished to a rounded biconvex end such that said second upper angled guide edge optimizes presentation area so as to protect tissue.

14. The device as claimed in claim 13, wherein said first lower guide edge, said second upper angled guide edge of said cutting head are configured such that when said trocar and said lower guide edge are inserted through a first surface said first lower guide edge penetrates said surface but said splayed second upper angled guide edge does not penetrate said surface, thereby capturing said surface below said second upper angled guide edge and above said first lower guide edge.

15. The cutting device as claimed in claim 1, wherein said trocar is constructed of a flexible metal wire.

16. The cutting device as claimed in claim 15, wherein said flexible metal wire is nitinol.

17. The cutting device as claimed in claim 1, wherein said trocar further comprises a trocar sub-assembly, said trocar subassembly is positioned within said housing so as to allow the user to manipulate said trocar to said first active position and said second retracted position.

18. The cutting device as claimed in claim 17, wherein said device further comprises a spring configured to bias against said trocar sub-assembly to force said trocar rearward into said second retracted position.

19. The cutting device as claimed in claim 18, wherein said trocar sub-assembly further comprises a trocar activation button extending out of the rear of said housing, allowing the user to place said trocar into said active position.

20. The cutting device as claimed in claim 19, wherein said trocar sub-assembly further comprises a trocar lock, said trocar lock being formed as a raised locking hook extending from said trocar base, such that said trocar lock catches on a latch formed from said housing and configured to configured to lock said trocar into said active position.

21. The cutting device as claimed in claim 19, wherein said trocar lock formed as a raised locking hook extending from said trocar base, is released from said latch, by depressing a plastically deformable trocar release area formed on said housing, allowing said trocar to return to said retracted position.

22. The cutting device as claimed in claim 19, wherein said trocar sub-assembly further comprises a trocar lock, said trocar lock being formed as a notch in said trocar base, such that said trocar lock catches on in a central opening of a locking button, said locking button being biased in an upward direction by a spring, said trocar lock configured to configured to lock said trocar into said active position

23. The cutting device as claimed in claim 22, wherein said trocar lock formed as a notch in said trocar base, is released from said central opening of a locking button, by depressing a release portion of said locking button, that extends out of an opening in said housing, downward against said spring, allowing said trocar to return to said retracted position.

24. A method for performing a subcutaneous incision is claimed, utilizing said cutting device from claim 1.

25. A method for performing a subcutaneous incision of the A1 pulley for trigger finger release is claimed, utilizing said cutting device from claim 1.

26. A cutting device for making subcutaneous incisions, said cutting device comprising:
a housing, said housing forming a handle;
a tubular blade sub-assembly extending from said housing said tubular blade sub-assembly maintaining a cutting head, said cutting head further having a first lower guide edge, a second upper angled guide edge, a cutaway on a first lateral side and a cutting edge on an opposite lateral side; and
a trocar, said trocar passing through said tubular blade sub-assembly, configured to be positioned in a first active position, with a tip of said trocar extending beyond the end of said cutting head and a second retracted position wherein said tip of said trocar is held within the end of cutting head.
